(19) United States
(12) Patent Application Publication
(10) Pub. No.: US 2018/0303092 A1
(43) Pub. Date: Oct. 25, 2018

(54) AMINE SALTS OF CARBOXYLIC ACID HERBICIDES

(71) Applicant: Taminco BVBA, Gent (BE)

(72) Inventors: Karen Mollet, Heusden (BE); Michael Schmidt, Norristown, PA (US); Kristof Moonen, Hamme (BE); Peter Roose, Deurle (BE); Jim Van Durme, Evergem (BE)

(73) Assignee: Taminco BVBA, Gent (BE)

(21) Appl. No.: 15/957,008

(22) Filed: Apr. 19, 2018

Related U.S. Application Data
(60) Provisional application No. 62/488,890, filed on Apr. 24, 2017.

(51) Int. Cl. A01N 39/04; C07C 215/08; C07C 65/21; C07C 211/11; C07C 59/70; C07C 211/07; C07C 209/68; C07C 51/41; C07C 213/08; A01N 37/40; A01N 25/02

(52) U.S. Cl. CPC A01N 39/04; C07C 215/08; C07C 65/21; C07C 211/11; C07C 59/70; A01N 25/02; C07C 209/68; C07C 51/412; C07C 213/08; A01N 37/40; C07C 211/07

(57) ABSTRACT

The invention generally involves combining specialty amines with herbicidal carboxylic acids to form a new generation of salts with improved characteristics. The salts contain a cation of an amine and an anion of a carboxylic acid herbicide. The amine is advantageously selected from mono-isobutylamine (MIBA), N-methylaminoethanol (MMEA), dimethylaminopropylamine (DMAPA), 2-dimethylaminoethanol (DMAE), methyldiethanolamine (MDEA), and 1,2-diaminopropane (1,2-DAP). The amine-herbicide combinations may possess one or more improved characteristics, including maximum loading, wettability, drift, viscosity, and volatilization.

### CROSS-REFERENCE TO RELATED APPLICATION

This application claims the benefit of U.S. Provisional Application No. 62/488,890 filed on Apr. 24, 2017 under 35 U.S.C. § 119(e)(1); the entire content of the provisional application is hereby incorporated by reference.

### FIELD OF THE INVENTION

The invention generally relates to the field of herbicides. The invention particularly relates to certain amine salts of herbicides, which, in free acid form, include at least one carboxylic acid moiety.

### BACKGROUND OF THE INVENTION

Various herbicide active ingredients have acidic functional groups in their molecular structure. When applied in an aqueous formulation, these acid groups can be neutralized with amines to obtain a formulation with the desired pH. Even though some amine-herbicide combinations are commercially available (e.g., ROUNDUP, BANVEL, etc.), they lack one or more desirable properties, including maximum loading (g acid/liter formulation), wettability, viscosity, volatility, log K_{ow} (influencing leaf penetration), and efficacy.

For example, efforts to solve the volatility problem of 2,4-D and dicamba, including preparation of water soluble salts (e.g., dimethylamine (DMA) salt of 2,4-D), have not been totally satisfactory, because upon volatilization of the amine, the herbicide reverts to its initial acid form, which has sufficient volatility to cause damage to sensitive crops.

Thus, there is a need for amine-herbicide combinations that possess one or more improved characteristics, such as maximum loading, wettability, drift, viscosity, and/or volatilization.

The present invention addresses these needs as well as others, which will become apparent from the following description and the appended claims.

### SUMMARY OF THE INVENTION

The invention is as set forth in the appended claims.

Briefly, in one aspect, the invention provides a salt comprising an acidic herbicide and a basic amine.

In one embodiment, the amine is selected from mono-isobutylamine (MIBA), N-methylaminoethanol (MMEA), methyldiethanolamine (MDEA), and dimethylaminopropylamine (DMAPA) and the herbicide is 2,4-dichlorophenoxyacetic acid (2,4-D).

In another embodiment, the amine is selected from MMEA, 2-dimethylaminoethanol (DMAE), DMAPA, methyldiethanolamine (MDEA), and 1,2-diaminopropane (1,2-DAP) and the herbicide is 3,6-dichloro-2-methoxybenzoic acid (dicamba).

In another aspect, the invention provides an herbicidal composition comprising an herbicidally effective amount of the salt and an agriculturally acceptable adjuvant or carrier.

In yet another aspect, the invention provides a method of making the salt, which comprises contacting the amine with the herbicide in water under conditions effective to form the salt.

In yet another aspect, the invention provides a method of controlling the growth of a plant. The method comprises the step of contacting the plant with an herbicidally effective amount of the herbicidal composition.

### BRIEF DESCRIPTION OF THE DRAWINGS

FIG. 1 shows a decision flow chart for preparing herbicide amine salt solutions.

FIG. 2 is an illustration of the wetting properties measurement technique used in the examples.

### DETAILED DESCRIPTION OF THE INVENTION

In general, the present invention provides amine salts of carboxylic acid herbicides, which, in free acid form, have at least one carboxylic acid moiety.

The amine salts are suitable for formulation into herbicidal application mixtures and/or concentrate compositions that exhibit acceptable stability and compatibility characteristics. The herbicide salts are suitable for preparing stable, highly loaded herbicidal solutions, concentrates, and/or emulsion concentrates. In accordance with various embodiments, the herbicidal amine salts can be more effective in reducing vapor drift from the evaporation of the herbicide compared to herbicide salts known in the art. In various embodiments, the inventive herbicidal amine salts can exhibit, simultaneously with low volatility, enhanced properties including one or more of maximum loading, wettability, drift reduction, and viscosity. In various embodiments, the inventive salts can enable the preparation of high-loaded herbicide formulations that are stable (chemically and physically), easy to manipulate (i.e., have appropriate viscosity properties), and readily soluble in water. In various embodiments, the inventive salts could also be less sensitive to volatilization and drift compared to commercially available herbicide salts and show better inherent wettability.

The herbicide salts of the invention comprise a carboxylate anion of a carboxylic acid herbicide. For example, in various embodiments, the herbicide salt may comprise the carboxylate anion of an herbicide selected from 3,6-dichloro-2-methoxybenzoic acid (dicamba) and 2,4-dichlorophenoxyacetic acid (2,4-D).

The herbicide salts of the invention further comprise a cation of various amine compounds. The amine compounds are advantageously selected from mono-isobutylamine (MIBA), N-methylaminoethanol (MMEA), dimethylaminopropylamine (DMAPA), 2-dimethylaminoethanol (DMAE), 1,2-diaminopropane (1,2-DAP), and methyldiethanolamine (MDEA).

The amine compounds may be in either the protonated or the quaternized form in the herbicide salts of the invention.

In one embodiment, the amine is MIBA and the herbicide is 2,4-D.

In another embodiment, the amine is MMEA and the herbicide is 2,4-D.

In yet another embodiment, the amine is DMAPA and the herbicide is 2,4-D.

In yet another embodiment, the amine is MMEA and the herbicide is dicamba.

In yet another embodiment, the amine is DMAE and the herbicide is dicamba.

In yet another embodiment, the amine is DMAPA and the herbicide is dicamba.

In yet another embodiment, the amine is 1,2-DAP and the herbicide is dicamba.

In yet another embodiment, the amine is MDEA and the herbicide is 2,4-D or dicamba.

Typically, an herbicide salt of the invention is derived from a carboxylic acid herbicide and a base amine compound. For example, in one method, herbicide in free acid form is mixed with an amine base in water or other suitable solvent. As recognized by those skilled in the art, formation of the herbicide salt results from proton exchange between the carboxylic acid moiety and the base.

Typically, when preparing the herbicide salts of the invention from a carboxylic acid herbicide containing a single carboxylic acid moiety and an amine compound containing a single amine functional group susceptible to forming a cation, equimolar or excess base may be used. However, when using some amine compounds that contain more than a single amine functional group (e.g., di- and tri-amines), equimolar or excess base compound may be unnecessary. With carboxylic acid herbicides containing more than one carboxylic acid moiety and/or amine compounds containing more than one amine functional group, the relative proportions of base compound and herbicide free acid can be adjusted as necessary.

Accordingly, in various embodiments, the molar ratio of the amine compound to the carboxylic acid herbicide is typically at least 0.4:1, at least 0.5:1, at least 0.6:1, at least 0.7:1, at least 0.8:1, at least 0.9:1, at least 1:1, at least 1.1:1, at least 1.2:1, at least 1.3:1, at least 1.4:1, at least 1.5:1, at least 1.6:1, at least 1.7:1, at least 1.8:1, at least 1.9:1, or at least 2:1. In these and other embodiments, the molar ratio of the amine compound to carboxylic acid herbicide may range from 0.4:1 to 2:1, from 0.5:1 to 2:1, from 0.7:1 to 2:1, from 0.8:1 to 1.8:1, from 1:1 to 2:1, from 1.2:1 to 1.8:1, from 0.5:1 to 1.5:1, or from 1:1 to 1.5:1.

Stated differently, equimolar or excess cations (i.e., protonated proton-accepting groups) may be provided when preparing the herbicide salts of the invention. Accordingly, in various embodiments, the molar ratio of cations to carboxylic acid herbicide anions (i.e., deprotonated proton-donating groups) is at least 1:1, at least 1.1:1, at least 1.2:1, at least 1.3:1, at least 1.4:1, at least 1.5:1, at least 1.6:1, at least 1.7:1, at least 1.8:1, at least 1.9:1, or at least 2:1. In these and other embodiments, the molar ratio of cations to carboxylic acid herbicide anions may range from 1:1 to 2:1, from 1:1 to 1.8:1, from 1.1:1 to 2:1, from 1.2:1 to 1.8:1, or from 1:1 to 1.5:1.

In certain instances, water-soluble herbicide salts are desirable so that aqueous herbicidal solutions or formulations can be prepared. Accordingly, in various embodiments, the herbicide amine salts of the invention are water soluble at room temperature or at elevated temperatures (e.g., 40-80° C.) such that they may be dissolved in an aqueous solution or formulated in an aqueous solution concentrate.

The herbicide salts of the invention may be used in the preparation of concentrates, tank-mixes, or ready-to-use (RTU) formulations. Tank-mix and RTU formulations comprising one or more of the herbicide salts of the invention typically comprise from 0.1 g a.e./L to 50 g a.e./L total herbicide loading, while concentrate formulations typically comprise from 50 to 1000 g a.e./L, from 300 to 1000 g a.e./L, from 350 to 1000 g a.e./L, from 400 to 1000 g a.e./L, from 450 to 1000 g a.e./L, or even from 500 to 1000 g a.e./L total herbicide loading. In various embodiments, for example, when the herbicide is dicamba, the concentrate formulations may comprise at least 700 g a.e./L, at least 800 g a.e./L, at least 850 g a.e./L, or at least 900 g a.e./L total herbicide loading. In various other embodiments, for example, when the herbicide is 2,4-D, the concentrate formulations may comprise at least 550 g a.e./L, at least 600 g a.e./L, at least 800 g a.e./L, or at least 850 g a.e./L total herbicide loading.

The herbicide salts of the invention may be formulated with conventional adjuvants, excipients, and/or additives. For example, the salts can be combined with a selection of adjuvants to enhance one or more of the salts' properties. Adjuvants are commonly used in agriculture to improve the performance of herbicides. Broadly defined, “an adjuvant is an ingredient that aids or modifies the action of the principal active ingredient.” The use of adjuvants with agricultural chemicals generally falls into two categories: (1) formulation adjuvants are present in the container when purchased by the dealer or grower; and (2) spray adjuvants are added along with the formulated product to a carrier such as water. The liquid that is sprayed over the top of a crop, weeds, or insect pest often will contain both formulation and spray adjuvants.

Formulation adjuvants may be added to the active ingredient for several reasons, including better mixing and handling, increased effectiveness and safety, better distribution, and drift reduction. These traits are accomplished by altering the solubility, volatility, specific gravity, corrosiveness, shelf-life, compatibility, or spreading and penetration characteristics. With the large number of formulation options available (solutions, emulsions, wettable powders, flowables, granules, and encapsulated materials), adjuvants can be advantageous in assuring consistent performance.

Spray adjuvants may be added to the tank to improve herbicide performance. Literally hundreds of chemical additives are now available that fall into this category. Spray adjuvants can be grouped into two broad categories: (1) activator adjuvants, including surfactants, wetting agents, stickers-spreaders, and penetrants; and (2) special purpose or utility modifiers, such as emulsifiers, dispersants, stabilizing agents, coupling agents, co-solvents, compatibility agents, buffering agents, antifoam agents, drift control agents, and nutritionals.

Other additives or ingredients may be introduced into the compositions of the present invention to provide or improve certain desired properties or characteristics of the formulated product. Thus, the herbicidal composition may further comprise one or more additional ingredients, such as surfactants, foam-moderating agents, preservatives or antimicrobials, antifreeze agents, solubility-enhancing agents, dispersants, stabilizers, dyes, and thickening agents. For example, in various embodiments, the herbicidal composition comprising an herbicidal salt of the invention, may further comprise a surfactant selected from the group consisting of alkoxylated tertiary etheramines, alkoxylated quaternaryetheramines, alkoxylated etheramine oxides, alkoxylated tertiary amines, alkoxylated quaternary amines, alkoxylated polyamines, sulfates, sulfonates, phosphate esters, alkyl polysaccharides, alkoxylated alcohols, and combinations thereof. The weight ratio of the carboxylic acid herbicide amine salt acid equivalent to surfactant can be readily determined by those skilled in the art (e.g., from 1:1 to 20:1, from 2:1 to 10:1 or from 3:1 to 8:1).

Application mixtures of the herbicides salts of the invention may be prepared by dissolving the salts in water or other suitable solvent or by suitable dilution of a concentrate composition and applying to the foliage of unwanted plants by methods known in the art. The application mixture can be applied to the foliage of a plant or plants at an application rate sufficient to give a commercially acceptable rate of weed control. This application rate is usually expressed as amount of herbicide per unit area treated, e.g., grams acid equivalent per hectare (g a.e./ha). Depending on plant species and growing conditions, the time required to achieve a commercially acceptable rate of weed control can be as short as a week or as long as three weeks, four weeks, or 30 days. Application mixtures of the herbicides salts can be applied before planting, at planting, pre-emergence, or post-emergence to crop plants depending on the particular herbicide salt and crop plant.

Application mixtures prepared with the herbicide salts of the invention may be applied to the foliage of crop plants and/or unwanted plants in the proximity of crop plants. Crop plants include hybrids, in-breeds, and transgenic or genetically modified plants having specific traits or combinations of traits including, without limitation, herbicide tolerance (e.g., tolerant to carboxylic acid herbicides or other herbicides), Bacillus thuringiensis (Bt), high oil, high lysine, high starch, nutritional density, and drought resistance. Particular crop plants include, for example, corn, peanuts, potatoes, soybeans, canola, alfalfa, sugarcane, sugar beets, peanuts, grain sorghum (milo), field beans, rice, sunflowers, wheat and cotton. In various embodiments, the crop plant is selected from the group consisting of soybeans, cotton, peanuts, rice, wheat, canola, alfalfa, sugarcane, sorghum, and sunflowers. In other embodiments, the crop plant is selected from the group consisting of corn, soybean, and cotton.

Herbicidal application mixtures containing an herbicide salt of the invention can be applied pre-planting of the crop plant, such as from two to three weeks before planting. The application mixture can be applied at planting, pre-emergence, or post-emergence to crop plants to control weeds in a field of the crop plants.

While not wishing to be bound by theory, volatility is a known problem of application mixtures containing salts of many carboxylic acid herbicides. Volatility of the acid herbicides is correlated to the free acid concentration in the aqueous solution. As the amine salting agent volatilizes from solution, the free acid concentration increases resulting in higher volatility of the herbicide. In some instances, the amine salts of the present invention could provide desirable low volatility through, for instance, increased amine molecular weight or hydrogen bond acceptance, keeping the amine in solution. A more stable amine concentration in solution results in reduced free acid herbicide in solution and reduced associated offsite movement.

The present invention includes and expressly contemplates any and all combinations of embodiments, features, characteristics, parameters, and/or ranges disclosed herein. That is, the invention may be defined by any combination of embodiments, features, characteristics, parameters, and/or ranges mentioned herein.

It is contemplated that any ingredient, component, or step that is not specifically named or identified as part of the invention may be explicitly excluded by at least some embodiments of the invention.

Any process, apparatus, compound, composition, embodiment, or component of the invention may be modified by the transitional terms “comprising,” “consisting essentially of,” or “consisting of,” or variations of those terms.

As used herein, the terms “acid equivalent,” “a.e.,” or “ae” refer to the amount of herbicide present without taking into account the weight of the counter-ion of the salt species present.

As used herein, the indefinite articles “a” and “an” mean one or more, unless the context clearly suggests otherwise. Similarly, the singular form of nouns includes their plural form, and vice versa, unless the context clearly suggests otherwise.

While attempts have been made to be precise, the numerical values and ranges described herein should be considered to be approximations (even when not qualified by the term “about”). These values and ranges may vary from their stated numbers depending upon the desired properties sought to be obtained by the present invention as well as the variations resulting from the standard deviation found in the measuring techniques. Moreover, the ranges described herein are intended and specifically contemplated to include all sub-ranges and values within the stated ranges. For example, a range of 50 to 100 is intended to describe and include all values within the range including sub-ranges such as 60 to 90 and 70 to 80.

Any two numbers of the same property or parameter reported in the working examples may define a range. Those numbers may be rounded off to the nearest thousandth, hundredth, tenth, whole number, ten, hundred, or thousand to define the range.

The content of all documents cited herein, including patents as well as non-patent literature, is hereby incorporated by reference in their entirety. To the extent that any incorporated subject matter contradicts with any disclosure herein, the disclosure herein shall take precedence over the incorporated content.

This invention can be further illustrated by the following examples of preferred embodiments thereof, although it will be understood that these examples are included merely for purposes of illustration and are not intended to limit the scope of the invention.

The data set forth in the following examples demonstrate that the herbicidal amine salts of the present invention can solve one or more of the following technical problems:
High loading of acidic functional groups in the concentrate without resulting in excessive viscosity at low temperatures. The product should be easy to dispense by pouring or pumping.
High-loaded concentrates being highly (physical and chemical) stable during storage.
High-loaded concentrates being easily diluted in water without the necessity of adjuvant addition.
Formed salts slowly releasing the active compound, thus ensuring an improved uptake and longer efficacy grade.
Increased efficacy of the herbicide due to synergetic interaction with the added amines in the concentrate formulation.
High compatibility of innovative concentrations with a wide of adjuvants, enabling further performance improvements.
Unique wettability properties of the innovative concentrates.

### EXAMPLES

### Salt Preparation

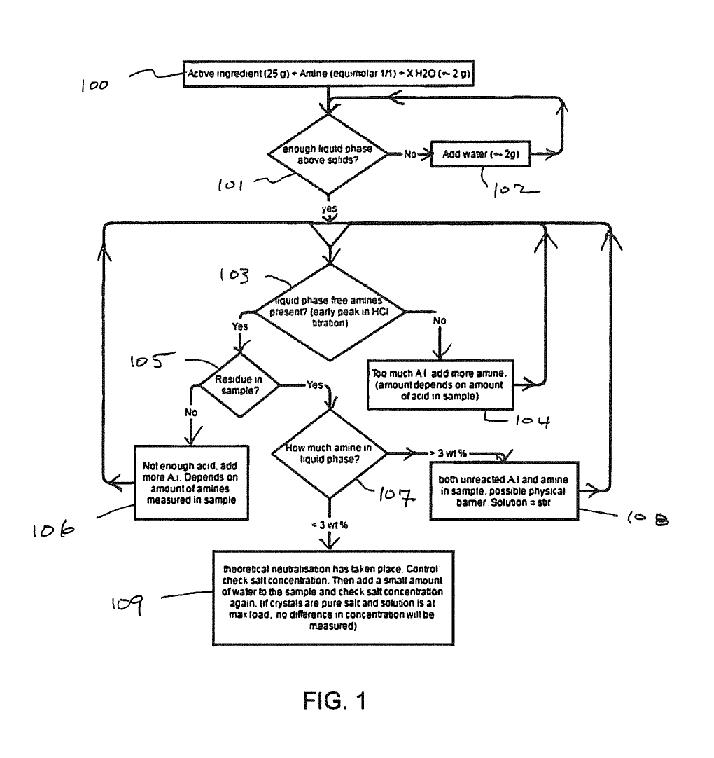

Neutralizing acidic herbicides (e.g., 2,4-D, dicamba, glyphosate, glufosinate) with amines can result in the formation of highly water soluble salts. However, practical inconveniences may occur when adding liquid amines to the solid herbicides, such as the formation of wetted powder, poorly mixed slurries, two apparently non-mixed layers, etc. To efficiently prepare a salt, the method, shown as a flowchart in FIG. 1, has been developed.

Referring to FIG. 1, step 100 involves using a mass balance to weigh 25 g of the active ingredient (a solid acidic herbicide, such as 2,4-D or dicamba) in a glass bottle. An equimolar amount of a basic amine (typically a liquid) is added to this glass bottle. Last, 2 mL of water is added. After adding the three compounds, the bottle is capped and placed on a shaking device for at least 12 hours.

After the mixing/shaking period, at step 101, the system/mixture is visually evaluated. If a solid or sludge is formed, step 102 is commenced. If a clear layer is present, step 103 is commenced.

In step 102, the glass bottle is opened and 2 mL of water is added. The bottle is capped again and placed on the shaking device for another 12 hours. Step 101 is then repeated.

At step 103, a small fraction of the liquid top layer is withdrawn to determine the free amine content. 0.1 N HCl is used as a titrant, and methanol is used as a solvent. Conductometry is used for detection. When analyzing samples, the designated amount of sample weighed should be around 100-400 mg, so that about 10-20 mL of the titrant is used. The result is given in an EP (equivalence point). The following calculation is used to obtain the amine wt %:

$$wt {\%}_{amine} = \frac{EP * {MM}_{amine}}{1000}$$

where MMₐₘᵢₙₑ is the molecular weight of the amine. If no amines are detected, go to step 104. If amines are detected, go to step 105.

The absence of amines in step 103 means that there is probably still unreacted acidic herbicide present in the system. To ensure that all herbicides are neutralized and converted into a salt, a small amount of amines (e.g., 5% of the original amount added) should be added at step 104. After shaking the enriched glass system for 12 hours, the liquid phase is reevaluated at step 103.

At step 105, a visual inspection of the system is conducted. The goal is to obtain a two-phase system containing salt solids and a saturated liquid top layer. If no salt solids are seen, then step 106 is performed. If salt solids can be seen, then step 107 is performed.

At step 106, if no salt solids are visible and free amines are still present, then additional acidic herbicide is introduced into the system (e.g., 5% of the original amount added). After shaking the enriched glass system for an additional 12 hours, the liquid phase is reevaluated at step 103.

At step 107, the amount of free amines in the liquid top layer is calculated based on the titration results. If the calculated amount is too high (e.g., ≥1, ≥2, or ≥3 wt %), then step 108 is performed. If the calculated amount is less than the desired amount (e.g., <1, <2, or <3 wt %), then step 109 is performed.

At step 108, the high amine concentration in the top layer may indicate that a complete neutralization reaction did not occurred. This may be due to poor mixing (e.g., due to crust formation) and that more intensive stirring is required (e.g., with a spoon, stir bar, or ultrasound). After physically breaking the crust and mixing the two phases, the system should be shaken for at least 12 hours. Then, step 103 is repeated.

At step 109, theoretical neutralization has taken place. The salt concentration can be quantified by titration. To confirm that the solid layer is solid salt and the top layer is a saturated (max. loading) solution, a small amount of water is added (the amounts of water should be small enough to avoid a complete dissolution of the solid salt). After re-equilibrium during at least 12 hours of shaking, and under the condition that two phases are still present, the salt concentration is determined again. If salt concentrations are identical as the original values, then it can be concluded that a maximum loaded solution was obtained.

The flow chart in FIG. 1 is based on (a) measuring the pH of the obtained liquid layer (too high a pH indicates the presence of unreacted amines) and/or (b) measuring the free amine content in the liquid. If neutralization is insufficient, increased stirring or placing the system is an ultrasonic bath may be required. These techniques should ensure mixing of the two reactants (amine and herbicide), until neutralization.

By using the decision flowchart, the following errors in making the salts can be avoided:
The evaluated liquid layer containing unreacted amines, thereby not fully neutralizing the added herbicide (due to poor mixing, formation of an impermeable salt layer, etc.).
The residual solid layer in the system containing unreacted herbicide, instead of salt crystals.

The resulting salt solution may contain suspended salt crystals. In which case, the solution can be deemed to be oversaturated.

### Analysis of the Max Load Solutions

To determine maximum loading of the salt in water, the method shown in the flowchart of FIG. 1 was used. The resulting sample contained at least two layers: a top layer of liquid and a bottom layer of solid salt. A water and amine determination was made of the top layer.

### Water Content

To check the water content, a well-established method of Karl-Fischer was used. This method gives an accurate wt % of H₂O in the sample.

### Amine Content

To determine the amine content, HCl-titration was used. HCl (0.1 N) was used as the titrant, and methanol was used as the solvent. 100-400 mg of the sample was taken from the liquid layer so that only 10-20 mL of the titrant was needed. The result was given in an EP. The following calculation was used to obtain the amine wt %:

$$wt {\%}_{amine} = \frac{EP * {MM}_{amine}}{1000}$$

where MMₐₘᵢₙₑ is the molecular weight of the amine.

In general, the sample's liquid phase only contains three components: water, free amine, and the herbicide salt. Therefore, the following calculation was used to determine the salt wt %:

wt %ₛₐₗₜ=100−wt %ₐₘᵢₙₑ−wt %_{water}

The results below are reported in g a.e./L (acid equivalent). This represents the amount in g of active herbicide in the sample (it does not represent the amount of functional acid groups on the herbicides molecule). The results were calculated using the following equation:

$${g}_{ae} / 1 = \frac{wt {\%}_{salt}}{{MM}_{acid} + ( {MM}_{amine} * F )} ( {MM}_{acid} ) * ρ * 1000$$

The MMs in the equation above are molecular weights (g/mol). ρ is the density of the liquid (g/mL), which may be measured using the density meter Anton Paar DMA 4500. F is a factor that takes into account the amount of base or acid groups on the amine or acid, respectively.

The g amine/L reported below was calculated using the following equation:

$${g}_{amine} / 1 = \frac{wt {\%}_{salt}}{{MM}_{acid} + ( {MM}_{amine} * F )} ( {MM}_{amine} * F ) * ρ * 1000$$

### Viscosity

The viscosity of the solutions prepared as above was determined using the Brookfield nr. 87 spindle. The applied speed depended on the viscosity of the sample. The torque applied was 50-80% of the machine's maximum. The results are expressed as mPa.

### Wetting Properties

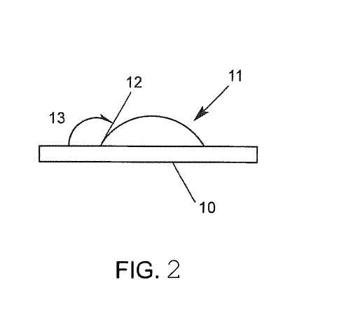

A visual test was used to determine the wetting property. FIG. 2 is an illustration of the test. A small droplet 11 of the max load product mentioned above was deposited by a syringe pointed vertically down onto a parafilm layer 10, which is hydrophobic. A profile picture of the droplet 11 was then captured by a camera. The approximate contact angle 13 of the droplet 11 with the parafilm layer 10 was then measured by eye using a protractor.

The wetting angle determines the surface energy or chemical affinity of the salt solution with the apolar reference material. A high wetting angle indicates a high chemical affinity, and thus a high degree of wetting of hydrophobic surfaces.

### Example 1

### Preparation of High-Loaded Dicamba Amine Salt Solutions

25.02 g of dicamba herbicide was mixed with 4.16 g of 1,2-DAP and 5.88 g of water. The ingredients were shaken for a minimum of 24 hours at room temperature (20° C.) to obtain an oversaturated salt solution (with precipitated salt crystals). Theoretically, 1 mol of dicamba can be neutralized using 0.5 mol of 1,2-DAP. Water was gradually added until all the salt crystals were dissolved to form a maximum loaded clear solution. The loading of the active ingredient was determined based on the amine content by HCl titration and the water content by Karl-Fischer.

Maximum loading of 803.2 g ae/L herbicide and 365.9 g amine/L were measured. The loadings are in line with a control salt made of DGA with dicamba.

Similar experiments were performed for other amines in combination with dicamba. The other amines and the results are shown in Table 1.

TABLE 1
Maximum Loadings of Dicamba Amine Salts

| Amine | | Max Load | |
|---|---|---|---|
| Name/Structure | Abbreviation | g ae/L | amine g/L |
| 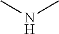 | DMA* | 1036.8 | 211.1 |
| dimethylamine | | | |
| 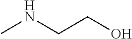 | MMEA | 928.8 | 357.7 |
| 2-methylaminoethanol | | | |
| 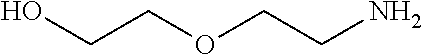 | DGA* | 854.9 | 435.5 |
| diglycolamine | | | |
| 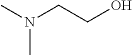 | DMAE | 849.0 | 408.3 |
| dimethylaminoethanol | | | |
| 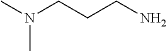 | DMAPA | 827.3 | 349.0 |
| dimethylaminopropylamine | | | |
| 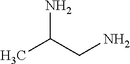 | 1,2-DAP | 803.2 | 365.9 |
| 1,2-diaminopropane | | | |
| 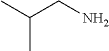 | MIBA | 713.7 | 475.0 |
| isobutylamine | | | |
| 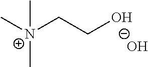 | Cbase | 481.5 | 308.5 |
| choline hydroxide | | | |

*Control

### Example 2

### Preparation of High-Loaded 2,4-D Amine Salt Solutions

Using the same methodology as described in Example 1, high-loaded 2,4-D salt solutions were prepared. The amines used and the results are shown in Table 2.

TABLE 2
Maximum loadings of 2,4-D Amine Salts

Max Load

| Amine | g ae/L | amine g/L |
|---|---|---|
| DMA* | 936.1 | 264.4 |
| DMAPA | 863.5 | 210.6 |
| MMEA | 774.3 | 302.5 |
| DMAE | 624.7 | 316.9 |
| MIBA | 497.8 | 183.5 |
| Cbase* | 195.0 | 315.6 |

*Control

As seen from Table 2, several 2,4-D amine salts have maximum loads that are comparable with the DMA control salt, while all were higher than the Cbase control salt.

### Example 3

Preparation of Amine Salt Solutions with Good Viscosity and Wetting Properties

Several amine herbicide formulations were prepared as described above in Examples 1 and 2. The viscosity of various formulations was measured using a Brookfield viscometer with nr.87 spindle at 20° C. Wettability was measured by visually measuring the contact angle after placing a standard droplet on a parafilm layer (hydrophobic). The results of dicamba amine salts are reported in Table 3.

TABLE 3
Viscosity and Wettability of Dicamba Amine Salt Concentrates

| | Viscosity | Wetting |
|---|---|---|
| Amine | (mPa) | Angle (°) |
| DMA* | 58.8 | 135 |
| MMEA | 3,967.0 | 120 |
| DGA* | 562.5 | 120 |
| DMAE | 184.4 | 115 |
| DMAPA | 1,791.0 | 120 |
| 1,2-DAP | 218.0 | 105 |
| MIBA | 46.1 | 130 |
| Cbase | 13.5 | 115 |

*Control

For dicamba, the DMA salt can be used as reference, since this is the salt used in BANVEL (a commercial product). From Table 3, it can be seen that the amine salts are defined by having relatively similar wetting properties, but vastly different viscosity properties.

Table 4 contains viscosity and wetting properties for 2,4-D amine salts.

TABLE 4
Viscosity and Wettability of 2,4-D Amine Salts

| | Viscosity | Wetting |
|---|---|---|
| Amine | (mPa) | Angle (°) |
| DMA* | 38.8 | 130 |
| DMAPA | 1,455.0 | 125 |
| MMEA | 21.2 | 110 |
| DMAE | 74.0 | 105 |
| MIBA | 60.1 | 155 |
| Cbase* | 12.4 | 145 |

*Control

As seen from the above results, the new salts according to the invention can have multiple advantages—resolving earlier described problems—compared to currently known/commercialized amine salts. An example of a salt according to the invention is 1,2-DAP salt of dicamba. This salt has the following combination of desirable characteristics:
a. Good viscosity properties (218.0 mPa), being intermediate between controls DMA salt (58.8 mPa) and DGA salt (562.5 mPa);
b. High maximum loading (803.2 g/L), being only slightly lower than controls DMA (1036 g/L) and DGA (854.9 g/L);
c. Higher hydrophilicity/reduced hydrophobicity: wetting angle of 105° vs. 135° (DMA) and 120° (DGA).

The invention has been described in detail with particular reference to preferred embodiments thereof, but it will be understood that variations and modifications can be effected within the spirit and scope of the invention.

We claim:

1. A salt comprising a cation of an amine and an anion of a carboxylic acid herbicide,
wherein the amine is selected from mono-isobutylamine (MIBA), N-methylaminoethanol (MMEA), and dimethylaminopropylamine (DMAPA), methyldiethanolamine (MDEA) and the herbicide is 2,4-dichlorophenoxyacetic acid (2,4-D); or
wherein the amine is selected from MMEA, 2-dimethylaminoethanol (DMAE), DMAPA, and 1,2-diaminopropane (1,2-DAP) methyldiethanolamine (MDEA) and the herbicide is 3,6-dichloro-2-methoxybenzoic acid (dicamba).

2. The salt according to claim 1, wherein the amine is MIBA and the herbicide is 2,4-D.

3. The salt according to claim 1, wherein the amine is MMEA and the herbicide is 2,4-D.

4. The salt according to claim 1, wherein the amine is DMAPA and the herbicide is 2,4-D.

5. The salt according to claim 1, wherein the amine is MMEA and the herbicide is dicamba.

6. The salt according to claim 1, wherein the amine is DMAE and the herbicide is dicamba.

7. The salt according to claim 1, wherein the amine is DMAPA and the herbicide is dicamba.

8. The salt according to claim 1, wherein the amine is 1,2-DAP and the herbicide is dicamba.

9. An herbicidal composition comprising an herbicidally effective amount of the salt according to any one of claims 1-8, and an agriculturally acceptable adjuvant or carrier.

10. The composition according to claim 1, which has a total herbicide loading of at least 550 g a.e./L.

11. The composition according to claim 10, which has a total herbicide loading of at least 700 g a.e./L.

12. The composition according to claim 11, which has a total herbicide loading of at least 800 g a.e./L.

13. The composition according to claim 12, which has a total herbicide loading of at least 900 g a.e./L.

14. A method of making a salt comprising contacting an amine with an herbicide in the presence of water under conditions effective to form the salt, wherein:
the amine is selected from mono-isobutylamine (MIBA), N-methylaminoethanol (MMEA), and dimethylaminopropylamine (DMAPA) and the herbicide is 2,4-dichlorophenoxyacetic acid (2,4-D); or
the amine is selected from MMEA, 2-dimethylaminoethanol (DMAE), DMAPA, and 1,2-diaminopropane (1,2-DAP) and the herbicide is 3,6-dichloro-2-methoxybenzoic acid (dicamba).

15. The method according to claim 14, comprising combining the amine, water, and herbicide under conditions effective to form a composition comprising a solid salt layer and a saturated liquid layer.

16. The method according to claim 15, comprising measuring the free amine content in the saturated liquid layer and optionally conducting steps to neutralize the composition after the measurement is taken.

17. A method of controlling the growth of a plant, the method comprising contacting the plant with an herbicidally effective amount of the composition according to claim 1 and an agriculturally acceptable adjuvant or carrier.

18. A salt comprising a cation of methyldiethanolamine (MDEA) and an anion of 2,4-dichlorophenoxyacetic acid (2,4-D) or 3,6-dichloro-2-methoxybenzoic acid (dicamba).

19. An herbicidal composition comprising an herbicidally effective amount of the salt according to claim 18, and an agriculturally acceptable adjuvant or carrier.

20. A method of making the salt comprising contacting an amine with an herbicide in the presence of water under conditions effective to form the salt, wherein amine comprises methyldiethanolamine (MDEA) and the herbicide comprises 2,4-dichlorophenoxyacetic acid (2,4-D) or 3,6-dichloro-2-methoxybenzoic acid (dicamba).
